(19) United States
(12) Patent Application Publication
(10) Pub. No.: US 2024/0344513 A1
(43) Pub. Date: Oct. 17, 2024

(54) VALVE COVER SYSTEM, VALVE COVER ASSEMBLY, AND METHODS OF USE

(71) Applicant: Steven K. Deel, Overland Park, KS (US)

(72) Inventor: Steven K. Deel, Overland Park, KS (US)

(21) Appl. No.: 18/628,746

(22) Filed: Apr. 7, 2024

Related U.S. Application Data
(60) Provisional application No. 63/458,866, filed on Apr. 12, 2023.

(51) Int. Cl. F04B 53/22; F04B 53/10; F04B 53/16; F16K 27/12

(52) U.S. Cl. CPC F04B 53/22; F04B 53/10; F04B 53/16; F16K 27/12

(57) ABSTRACT

A valve cover system, a valve cover assembly, and method of use are presented. The valve cover system and assembly forming part of a reciprocating pump or mud pump. Mud pumps and/or reciprocating pumps are used, often as part of a drilling set up. Mud pumps come in a variety of sizes, shapes, and designs. The valve cover system and assembly presented provides increased safety during installation and servicing as well as improvements in properly tightening and ensuring proper tightening of the assembly for high pressure operation. The present disclosure reduces manufacturing cost, maintenance costs, and the like through the design presented. The present disclosure presents a seal retainer and valve cover assembly for use in a reciprocating pump fluid end having jack bolts and a jack plate.

### CROSS-REFERENCE TO RELATED APPLICATIONS

The present application claims priority to the U.S. Provisional Patent Application No. 63/458,866 which was filed on Apr. 11, 2023, which is hereby incorporated by reference herein in its entirety, including any figures, tables, or drawings.

### FIELD OF THE DISCLOSURE

This disclosure relates to a valve cover system, a valve cover assembly, and method of use. More specifically, and without limitation, the present disclosure relates to a system, assembly, and method of assembly and disassembly for a high pressure operation and valve cover. More specifically, and without limitation, the present disclosure relates to a valve cover system and assembly which improves upon the safety and ease of installing and removing valve covers and the various components of a valve cover and/or installing valve covers of reciprocating pumps and other high pressure machines.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document. Copyright Blue Iron Engineering LLC. All rights reserved.

### BACKGROUND OF THE DISCLOSURE

Pressurized pumping systems are well known in the art. Pressurized pumps are the predominant fuel-pumping method used in the art today. As one example, and used herein for ease of explanation and application, reciprocating pumps are utilized in the drilling process to deliver high pressure fluid for fuel extraction. These pumps, also referred to as mud pumps, may be single or multi-cylinder pumps and/or may also refer to as multiplex pumps, such as those found in fracking applications. Said another way, the drilling or oil and gas wells used in drilling involve and require the use of large reciprocating pumps generally referred to as mud pumps. These pumps provide high pressure drilling fluid also known as drilling mud, to the well bore.

High pressure pumps, or mud pumps, are typically composed of two sections and/or two ends, a power end and a fluid end. The fluid end of a mud pump houses valves which separate high and low pressure areas within the fluid end. The fluid end block also have several openings through which a number of valves are installed and/or removed for maintenance. In order to seal these openings, valve covers, which are durable to the high pressure operations are installed on the fluid end, to contain the high pressure fluid. Traditional valve cover designs for mud pumps consist of the following components: Seal, Seal Retainer (plug), Threaded ring, and a screw gland.

Traditional valve cover assembly involves placing the seal in the fluid end bore and installing the seal retainer on top of the seal. The screw gland is then installed by threading it into the threaded ring until the screw gland comes into contact with the seal retainer. An installer then places a steel bar through open holes in the screw gland to further tighten the gland against the seal retainer. This heavy operation requires a sledge hammer to further tighten the screw gland against the seal retainer. Removal of the screw gland after operation then also requires many hits and extensive force of a sledge hammer or similar. This can be very dangerous and often results in injury or extensive work for those installing and/or removing the screw gland.

From a safety standpoint, the use of a sledge hammer to tighten the assembly has resulted in injuries due to a misplaced strike or material, chips being thrown from the metal bar being hit, etc. To properly tighten a screw gland in today's operations is very difficult as pressures in modern pumps have increased from 5000 psi to 7500 psi in many applications.

Thus, there is a long-felt need in the art for a valve cover system, a valve cover assembly, and method of use which improves upon the safety of the state of the art, and provides ease of use for a complex and heavy duty operation. The present disclosure provides a safer means of valve cover assembly and disassembly for installation and/or maintenance and/or removing. Said another way, the present disclosure provides for increased safety and ease of operation of a mud pump or multiplex pump or similar. The present disclosure also provides for ensuring that the valve cover is properly secured prior to activating high pressure operations. Furthermore, the present disclosure also provides reduced manufacturing costs. Furthermore, the present disclosure also provides for reduced labor and maintenance costs. These and other contributions and considerations provided herein provide the state of the art with a more cost effective, safe, and easy to use valve cover assembly.

The disclosure herein provides these advantages and others as will become clear from the specification and claims provided.

### SUMMARY OF THE DISCLOSURE

A valve cover system, a valve cover assembly, and method of use are presented. More specifically, and without limitation, the present disclosure provides a safe and easy to use valve cover system, assembly, and method of assembly and disassembly for a high pressure operation and valve cover. Said another way, and without limitation, the present disclosure provides the state of the art with a valve cover system and assembly which improves upon the safety and ease of installing and removing valve covers and the various components of a valve cover and/or installing valve covers of reciprocating pumps and other high pressure machines.

Thus, it is a primary object of the disclosure to provide a valve cover system, a valve cover assembly, and method of use that improves upon the state of the art.

Another object of the disclosure is to provide a valve cover system, a valve cover assembly, and method of use that is safe to use.

Yet another object of the disclosure is to provide a valve cover system, a valve cover assembly, and method of use that is easy to use.

Another object of the disclosure is to provide a valve cover system, a valve cover assembly, and method of use that requires less force for assembly.

Yet another object of the disclosure is to provide a valve cover system, a valve cover assembly, and method of use that requires less effort for disassembly.

Another object of the disclosure is to provide a valve cover system, a valve cover assembly, and method of use that is accurate in sealing.

Yet another object of the disclosure is to provide a valve cover system, a valve cover assembly, and method of use that is quick and efficient.

Another object of the disclosure is to provide a valve cover system, a valve cover assembly, and method of use that does not require a sledge hammer for removal or assembly.

Yet another object of the disclosure is to provide a valve cover system, a valve cover assembly, and method of use that is quick to assemble, quick to disassemble and efficient in time and effort.

Another object of the disclosure is to provide a valve cover system, a valve cover assembly, and method of use that is robust.

Yet another object of the disclosure is to provide a valve cover system, a valve cover assembly, a valve cover assembly, and method of use that saves time in maintenance and assembly.

Another object of the disclosure is to provide a valve cover system, a valve cover assembly, and method of use that is high quality.

Yet another object of the disclosure is to provide a valve cover system, a valve cover assembly, and method of use that is efficient with higher operating pressures of current mud pump operations.

Another object of the disclosure is to provide a valve cover system, a valve cover assembly, and method of use that provides an improved valve cover assembly.

Yet another object of the disclosure is to provide a valve cover system, a valve cover assembly, and method of use that provides an improved seal retainer.

Another object of the disclosure is to provide a valve cover system, a valve cover assembly, and method of use that does not require specialized tooling.

Yet another object of the disclosure is to provide a valve cover system, a valve cover assembly, and method of use that is relatively simple in design.

Another object of the disclosure is to provide a valve cover system, a valve cover assembly, and method of use that provides ease of assembly and disassembly due to the relatively simple design.

Yet another object of the disclosure is to provide a valve cover system, a valve cover assembly, and method of use that is cost effective.

Another object of the disclosure is to provide a valve cover system, a valve cover assembly, and method of use that can be installed with basic hand tools.

Yet another object of the disclosure is to provide a valve cover system, a valve cover assembly, and method of use that can be removed with basic hand tools.

Another object of the disclosure is to provide a valve cover system, a valve cover assembly, and method of use that is mechanically efficient.

Yet another object of the disclosure is to provide a valve cover system, a valve cover assembly, and method of use that is mechanically efficient, offering a 66:1 mechanical advantage in one embodiment.

Another object of the disclosure is to provide a valve cover system, a valve cover assembly, and method of use that is mechanically efficient, offering a 66:1 mechanical advantage in one embodiment, achieved by torquing a plurality of cap screws as opposed to a large diameter coarse thread as is traditional.

Yet another object of the disclosure is to provide a valve cover system, a valve cover assembly, and method of use that is mechanically superior to the existing state of the art, requiring only 150 ft-lbs of torque per cap screw for installation which then generates 10,000 or more ft-lbs of torque on the screw gland. In this way, the effort or strain of the traditional screw gland is not transferred to the installer or maintenance crew.

Another object of the disclosure is to provide a valve cover system, a valve cover assembly, and method of use that reduces manufacturing costs.

Yet another object of the disclosure is to provide a valve cover system, a valve cover assembly, and method of use that reduces operating costs.

Another object of the disclosure is to provide a valve cover system, a valve cover assembly, and method of use that reduces maintenance costs due to ease of installation and disassembly.

Yet another object of the disclosure is to provide a valve cover system, a valve cover assembly, and method of use that can be implemented on mud pumps.

Another object of the disclosure is to provide a valve cover system, a valve cover assembly, and method of use that is suitable for various pumps used in fracturing operations.

Yet another object of the disclosure is to provide a valve cover system, a valve cover assembly, and method of use that can be implemented in a variety of systems such as twist lock applications, wedge applications and other fluid end block applications.

These and other objects, features, or advantages of the present disclosure will become apparent from the specification and claims.

### BRIEF DESCRIPTION OF THE DRAWINGS

The drawings accompanying and forming part of this specification are included to depict certain aspects of the disclosure.

FIG. 1 is a top perspective view of a reciprocating pump or mud pump; the view showing the mud pump having a power end and a fluid end; the view showing a high pressure fluid end; the view showing a low pressure area; the view showing a plurality of blocks; the view showing a plurality of openings; the view showing a bore; the view showing a plurality of valves.

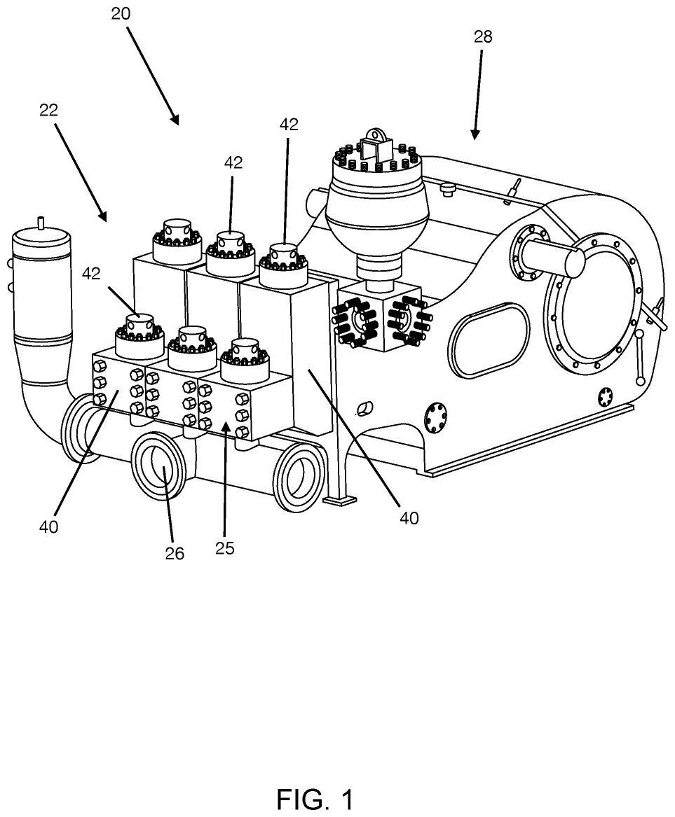

FIG. 2 is a cross sectional view showing a valve; the view showing a screw gland; the view showing a seal retainer plug.

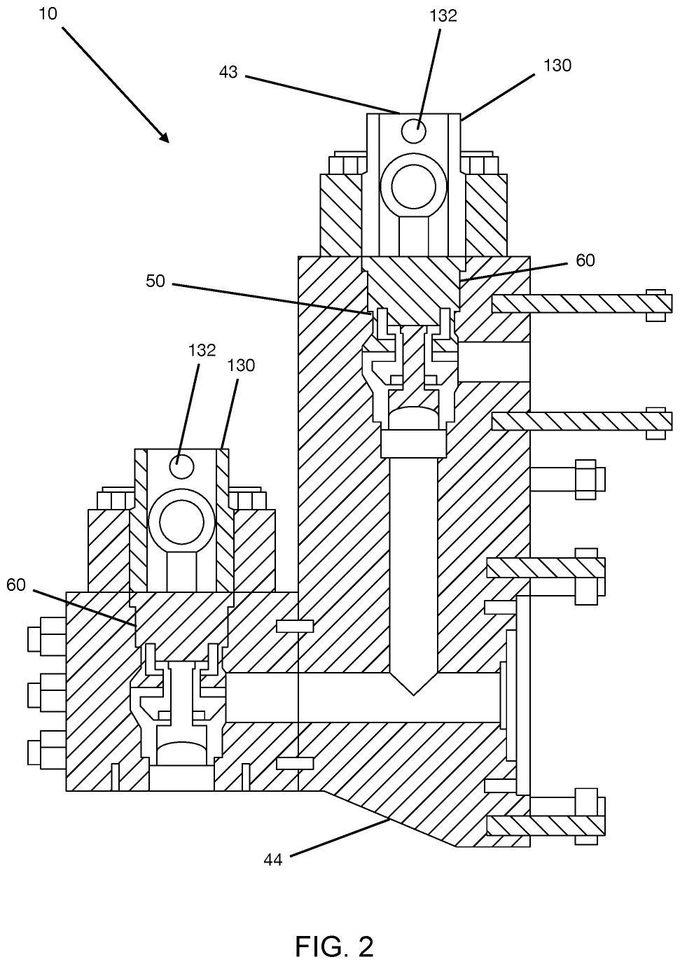

FIG. 3 is a cross sectional view showing a valve; the view showing a seal plug; the view showing a stem.

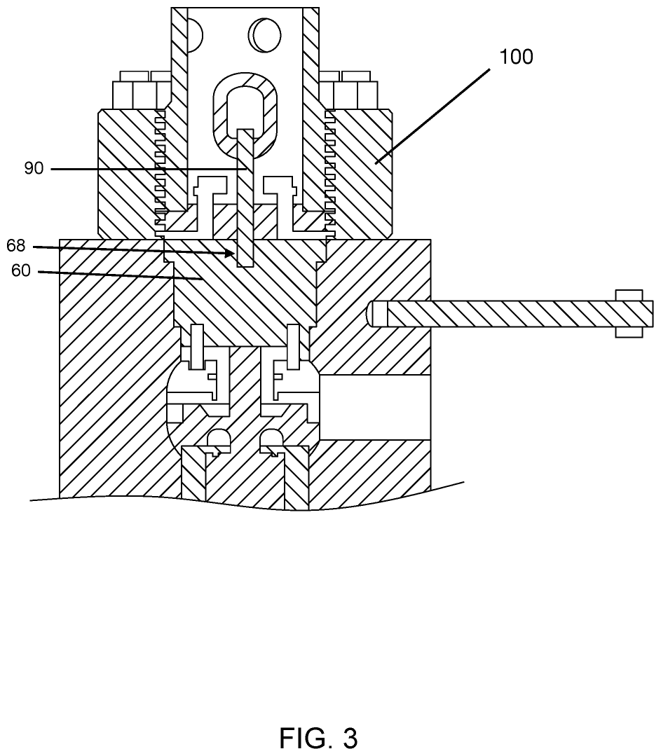

FIG. 4 is a top perspective view; the view showing a valve cover; the view showing a stem; the view showing a lift eye; the view showing a plurality of jack bolts; the view showing a jack plate.

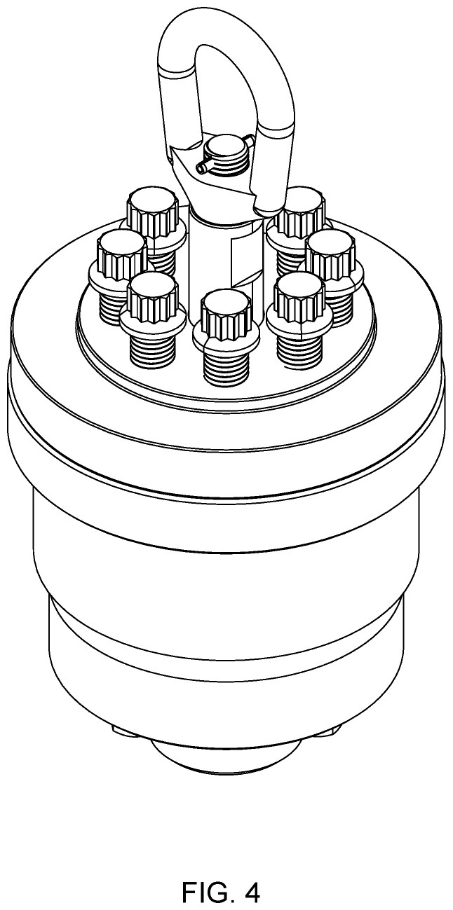

FIG. 5 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing a seal.

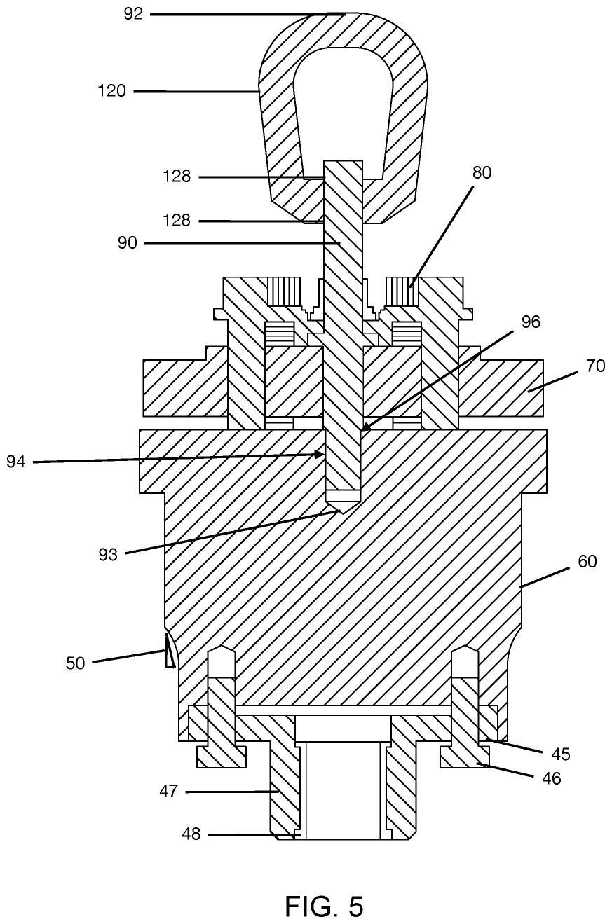

FIG. 6 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing a threaded ring.

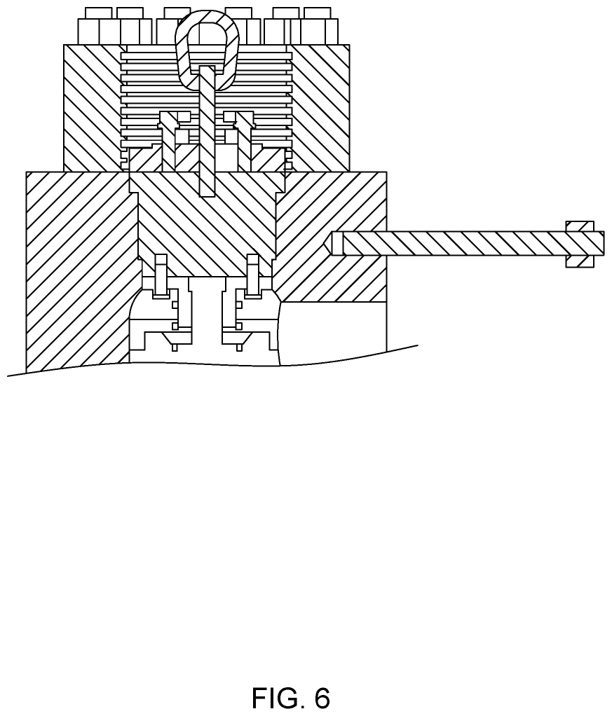

FIG. 7 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing a threaded ring; the view showing a screw gland; the view showing the screw gland having a plurality of apertures.

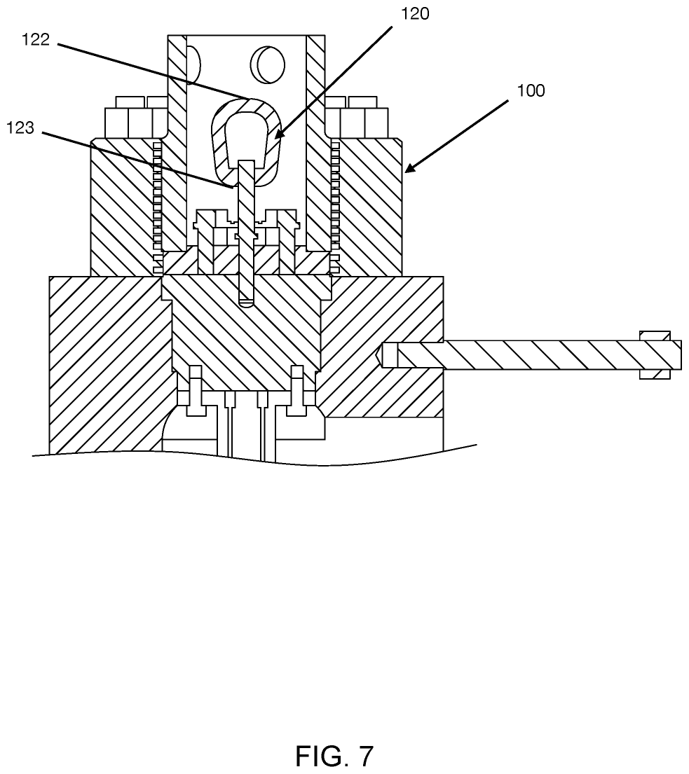

FIG. 8 is a top view of a valve cover; the view showing a stem; the view showing a lift eye; the view showing a jack plate; the view showing a plurality of jack bolts.

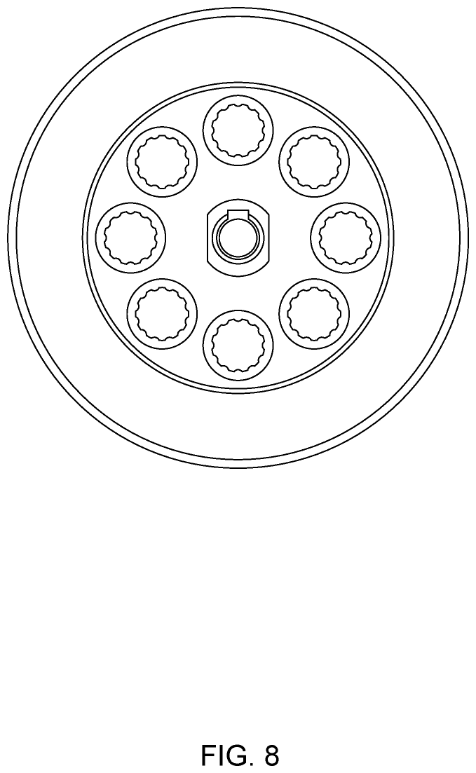

FIG. 9 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing a threaded ring; the view showing a screw gland; the view showing the screw gland having a plurality of apertures.

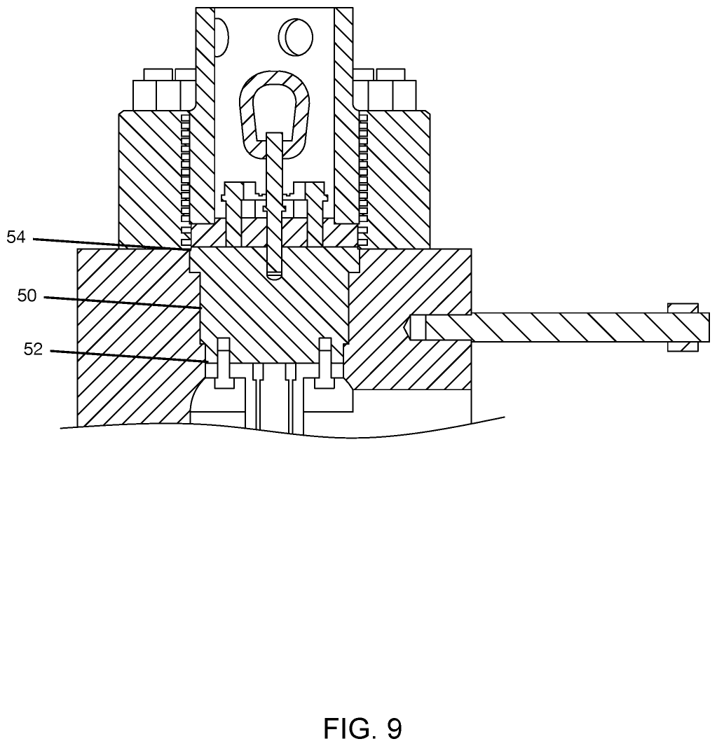

FIG. 10 is a view showing a plurality of valves and/or fluid ends of a mud pump; the view showing a plurality of hand tools engaged with the valve covers.

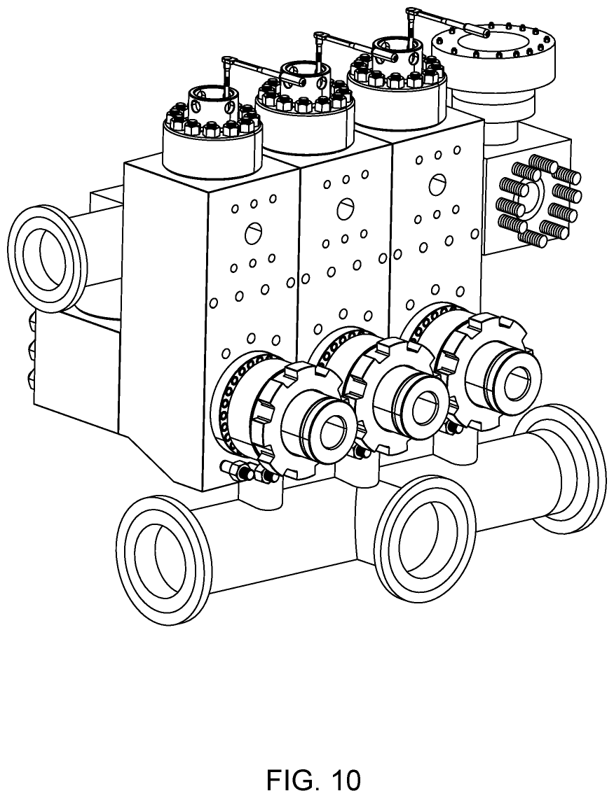

FIG. 11 is a view showing a plurality of valves and/or fluid ends of a mud pump; the view showing a plurality of hand tools engaged with the valve covers.

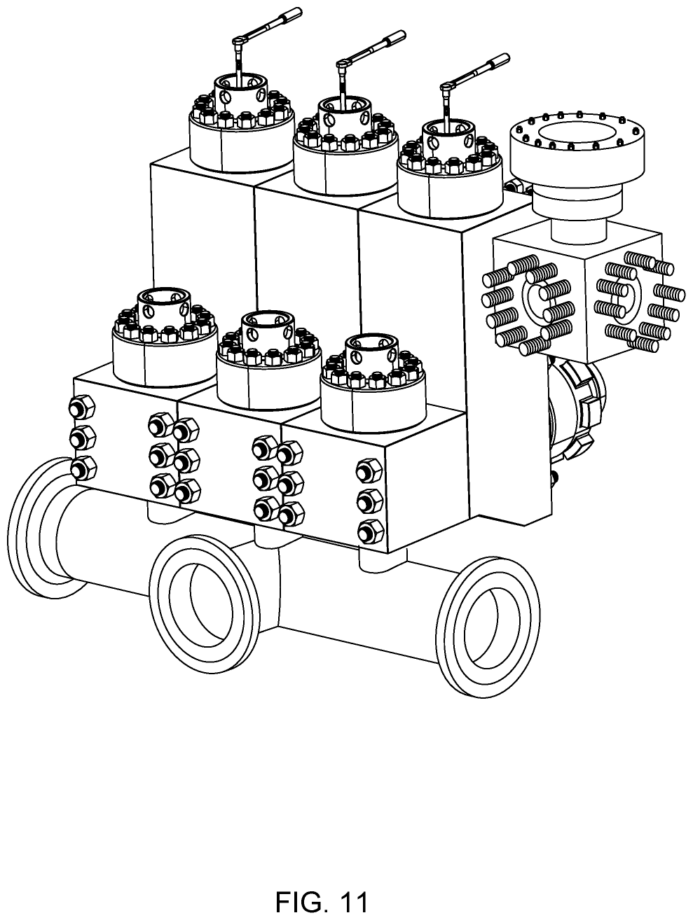

FIG. 12 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing the screw gland having a plurality of apertures.

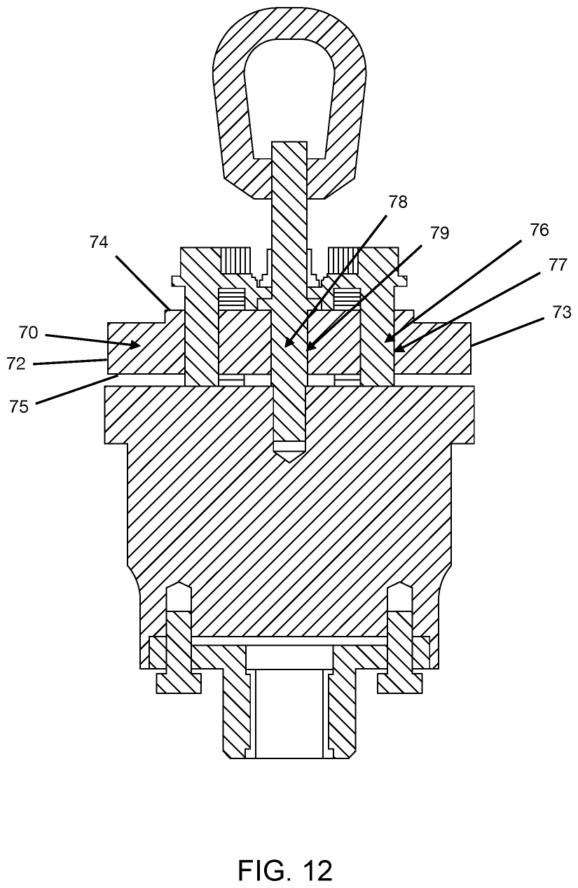

FIG. 13 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing the screw gland having a plurality of apertures.

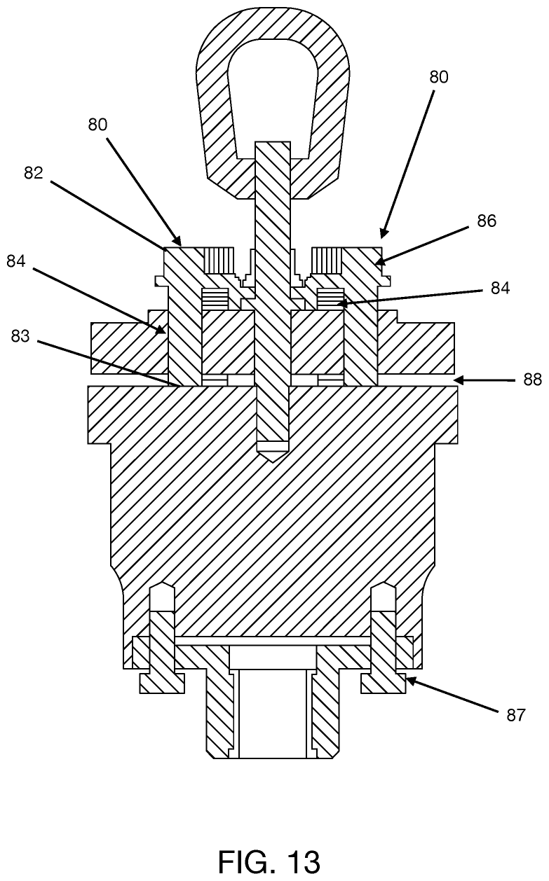

FIG. 14 is a cross sectional view; the view showing a valve cover; the view showing a stem; the view showing a lift eye; the view showing a plurality of jack bolts; the view showing a jack plate; the view showing cap screws; the view showing the valve guide and the valve guide retainer; the view showing a threaded ring; the view showing a screw gland; the view showing the screw gland having a plurality of apertures.

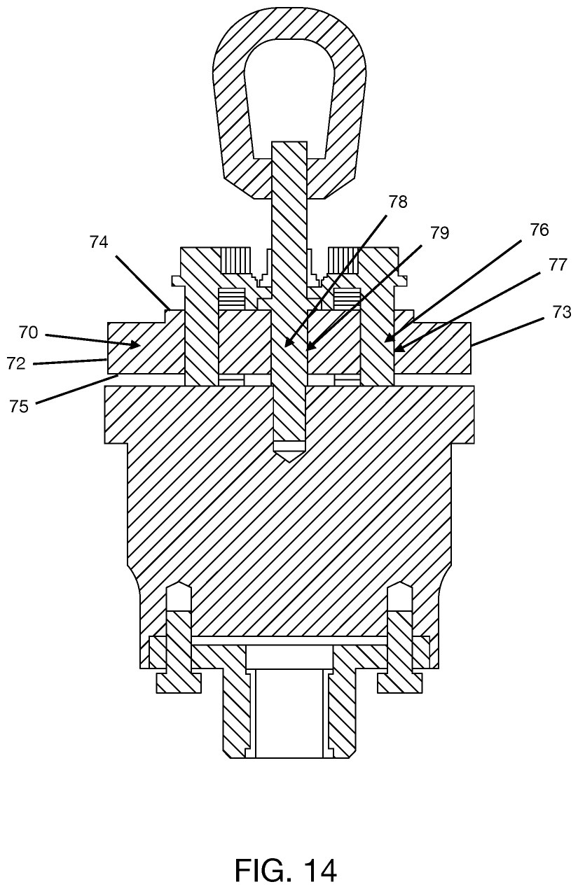

### DETAILED DESCRIPTION OF THE DISCLOSURE

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

### System:

With reference to the figures, a valve cover system, a valve cover assembly, and method of use 10 are presented. valve cover system, a valve cover assembly, and method of use 10 (hereafter referred to as “valve cover system”, “valve cover assembly”, “valve cover system”, “seal cover system”, or simply “system”) is formed of any suitable size, shape and design.

The present disclosure includes a threaded ring, a screw gland, and a seal (as will be further described herein). The present seal retainer is a replacement of traditional seal retainers and functions in an entirely novel way. The present seal retainer assembly includes a jack plate and a plurality of jack bolts (to be further described herein). The lower end of the center stem (to be further described herein) passes through the jack plate and threads into the seal retainer to maintain concentricity between the seal retainer and the jack plate. The lower shoulder on the stem serves as a mechanical stop to limit axial movement of the jack plate within the assembly.

Installation/Assembly: In this way, the present disclosure is novel because the present disclosure does not require a sledge hammer and/or large dangerous force of a user to assemble or disassemble. Alternatively to traditional designs and teaching away from adding force required for assembly or disassembly, the present disclosure provides a valve cover assembly 10 which seals with an improved seal due to the plurality of jack bolts located in the jack plate. In one embodiment, as shown herein, the jack bolts located in the jack plate are unthreaded such that the jack plate sits flush on top of the seal retainer.

Once the jack plate is flush on the seal retainer, the screw gland is threaded in by hand into the threaded ring. The screw gland then engages the top surface of the jack plate, without requiring a sledge hammer. Once the screw gland is installed and is in contact with the jack plate, the plurality of cap screws are tightened evenly in a cross-pattern (to be further described herein. In one example, the cap screws are tightened to a predefined torque.

The tightening and/or engagement of the cap screws in this way creates an axial force which in turn creates an axial (separating) force which places the seal retainer in compression against the fluid block. With the jack bolts torqued to the predefined value, the fluid end is now sealed and capable of high pressure operation.

Disassembly/Maintenance: The disassembly and/or disengagement and/or maintenance of an already installed system is the inverse of the above. Once the system is shut down and pressure removed, the plurality of jack bolts can be un-torqued from the predefined value in the reverse or in any relative order for easy removal and/or loosening of the assembly. The assembly, system, and methods of use will be further described herein.

In the arrangement shown, as one example, valve cover system 10 includes, in the embodiment(s) depicted, a user 12, a well 14, a high pressure fluid 16, a mud pump 20, a valve 40 or plurality of valves 40, a seal 50, a seal retainer 60, a jack plate 70, a plurality of jack bolts 80, a stem 90, a threaded ring 100, a lift eye 120, and a screw gland 130, among other components, features, and functionality.

### User (or Plurality Thereof):

In the arrangement shown, as one example, system 10 includes a user 12. User 12 may be any user interacting with or utilizing the system 10. This may include viewing, assembling, installing, disassembling, performing maintenance on, controlling, analyzing, manipulating, and/or interacting with system 10. Users are not limited to a single user but may be a plurality of users and/or may also be referred to as a crew or maintenance crew/staff.

### Well:

In the arrangement shown, as one example, system 10 may also include a well 14 (also referred to as “Well Bore”, “Oil Well”, “Gas Well”, or simply “Well”). Well may also be considered a drilling rig or similar overall structure. The applications, as will become apparent herein, may be part of a land-based right, a mobile drilling rig, or similar. Furthermore, the present disclosure is applicable or readily adaptable to any type of drilling rig with a pump and/or other supporting elements. Additional applications may include, but are not limited to, jack-up rigs, semisubmersibles, drills, drill ships, coil tubing rigs, well service rigs adapted for drilling and/or re-entry operations, and casing drilling rigs, among others within the scope of the present disclosure.

In one example, a drilling rig and/or well may include a mast, a rig floor, a crown block, a traveling block, a crown block being located at or near the top of the mast, a traveling block, the traveling block hanging from m the crown block by a drilling line, the drilling line extending from a lifting gear to drawworks for reeling in and/or out to cause the traveling block to be lowered and/or raised, an anchor, and the like as are commonly found among traditional rigs/wells. Finally, the rig and/or well may include a mud pump system.

### Mud Pump:

In the arrangement shown, as one example, system 10 includes a mud pump 16 (also referred to as “reciprocating pump”, “multiplex pump”, or simply “pump”). Pump 16 is formed of any suitable size, shape, and design, and is configured to provide high pressure fluid 18 to a well at a well bore.

In the arrangement shown, as one example, the reciprocating pump 16 includes a fluid end 22—having a high pressure area 23 and a low pressure area 24—a plurality of blocks 25, a plurality of openings 26, and a bore 27, and a power end 28, among other components and functionality.

In the arrangement shown, as one example, multiplex pump 16 is configured to receive the high pressure fluid (also known as “drilling fluid”, or “mud”), oftentimes from a tank, and deliver the high pressure fluid to the string through a hose or other conduit. In the present disclosure, the exemplary systems disclosed, by example and for ease of explanation are mainly the fluid end and in particular the valve cover assembly 10 of the fluid end 22 of the mud pump 16. The fluid end may also include an intake, a discharge, and other features. The high pressure of the fluid end requires a valve cover assembly.

### Valves:

In the arrangement shown, as one example, system 10 includes a valve 40 or valve cover 42 (also referred to as “valve cover assembly”, or simply “cover”). Valve cover 42 is formed of any suitable size, shape, and design, and is configured to be attached to the fluid end of the mud pump so as to provide access to the fluid end for maintenance and the like. The valve cover 42 must be suitable for withstanding the high pressures under which the fluid end operates.

In the arrangement shown, as one example, the valve cover 42 includes a top 43, a bottom 44, a lock washer 45, a cap screw 46, a valve guide retainer 47, and a valve guide 48, among other components, features, and functionality. When these components are engaged, a seal is formed.

### Seal/Seal Retainer:

In the arrangement shown, as one example, system 10 includes a seal retainer 50/60 (also referred to as “Retainer”, “Plug”, or simply “Seal”). Seal 60 is formed of any suitable size, shape, and design, and is configured to provide a fluid tight seal at the fluid end of the module. The seal is important in operation because intense pressure is applied to the seal and the seal must hold in order to maintain operation and safety. Said another way, the plug of the valve cover assembly is placed so that during operation (compression and expansion), the high pressure fluid is contained within the fluid end. Thus, the seal experiences intense pressures during operation.

In the arrangement shown, as one example, the seal retainer extends a length 51, includes a top 52, a bottom 54, and a plurality of threads 68 (for receiving the stem, to be further described herein).

### Jack Plate:

In the arrangement shown, as one example, system 10 includes a jack plate 70 (also referred to as simply “plate”). Plate 70 is formed of any suitable size, shape, and design, and is configured to hold the seal retainer in place, hold the stem in place, and the like.

In the arrangement shown, as one example, the jack plate extends a length 71 from a first end 72 to a second end 73 between a top 74 and a bottom 75. Furthermore, the jack plate 70 also includes a plurality of apertures 76 for receiving the jack bolts (to be further described herein). In one example, and in the arrangement shown herein, the plurality of apertures 76 are threaded with threads 77. Furthermore, and in the arrangement shown, as one example, the jack plate 70 includes a stem aperture 78 for receiving the stem 90 (to be further described herein); the stem aperture having a plurality of threads 79 (to be further described herein).

### Plurality of Jack Bolts:

In the arrangement shown, as one example, system 10 includes a plurality of jack bolts 80 (also referred to as “jack plate bolts”, “jack bolts”, or simply “bolts”). Jack bolts 80 are formed of any suitable size, shape, and design, and are configured to tighten the seal retainer and provide a safe and efficient means of tightening and/or securing the seal retainer so as to make removal and maintenance and/or engagement easier.

In the arrangement shown, as one example, the present disclosure includes a threaded ring, a screw gland, and a seal. The seal retainer includes the operation of the jack plate 70 and jack bolts 80. The jack bolts are configured to engage or disengage the seal retainer. Furthermore, the seal retainer includes a center stem which passes through the jack plate and threads into the seal retainer to maintain concentricity between the seal retainer and the jack plate. The lower shoulder on the stem serves as a mechanical stop to limit axial movement of the jack plate within the assembly. With concentricity in place, the jack bolts tighten and secure the seal retainer in association with the jack plate.

In the arrangement shown, as one example, the jack bolts are configured in the jack plate and are unthreaded such that the jack plates sits flush on top of the seal retainer. The screw gland is then threaded by hand until it lands on the top surface of the jack plate. Once the screw gland is in place and in contact with the jack plate, the cap screws of the plurality of jack bolts 80 are tightened evenly in a cross pattern until the predefined torque is achieved. Tightening the cap screws of the plurality of jack bolts results in an axial force which in turn creates an axial (separating) force 88,89 which places the seal retainer in compression against the fluid end block. In this way, the fluid end of the mud pump is sealed and capable of high pressure operation.

In the arrangement shown, as one example, each of the plurality of jack bolts extends a length 81 from a top 82—where the cap 86 and cap screw 87 are located—to a bottom 83. Each of the plurality of jack bolts 80 also includes threads 84.

In the arrangement shown, as one example, 8 jack bolts 80 are utilized. However, other numbers of jack bolts are hereby contemplated for use. Alternative numbers of jack bolts may be utilized depending on the size, pressure, and type of application, and/or a combination thereof. For example, one jack bolt might be used, two jack bolts are contemplated for use, three jack bolts are contemplated for use, four jack bolts, five jack bolts, six jack bolts, seven jack bolts, nine jack bolts, or more.

### Stem:

In the arrangement shown, as one example, system 10 includes a stem 90 (also referred to as “concentric device”, or simply “stem”). Stem 90 is formed of any suitable size, shape, and design, and is configured to provide concentricity and fit through the jack plate and thread into the seal retainer.

In the arrangement shown, as one example, the stem 90 extends a length 91 from a first end or top end 92 where a lift eye is secured to a second end or lower end 93 which includes threads 94 and threads into the seal retainer. Stem 90 also includes a lower shoulder 96 which rests on and/or engages with the jack plate.

### Threaded Ring:

In the arrangement shown, as one example, system 10 includes a threaded ring 100. Threaded ring 100 is formed of any suitable size, shape, and design, and is configured to receive the screw gland.

In the arrangement shown, as one example, a threaded ring is shown for ease of explanation and is used in this application. However, alternative applications of the present disclosure are hereby contemplated for use. Alternative applications include, but are not limited to a twist lock, a wedge, and/or other means of fastening the gland to the fluid end block.

### Lift Eye:

In the arrangement shown, as one example, system 10 includes a lift eye 120 (also referred to simply as “eye”). Lift eye 120 is formed of any suitable size, shape, and design, and is configured to provide an attachment to the stem. In the arrangement shown, as one example, the lift eye forms a loop or semi-circle which can be secured for lifting and/or placement. Furthermore, the lift eye includes retaining rings such that the lift eye can be secured and/or removed from the stem. Furthermore, the lift eye can be utilized to twist and/or torque the stem into place; via engaging and/or disengaging.

### Screw Gland:

In the arrangement shown, as one example, system 10 includes a screw gland 130. Screw gland 130 is formed of any suitable size, shape, and design, and is configured to maintain the jack plate and serve as a redundancy keeping the jack plate in place. The screw gland includes a plurality of apertures for assistance in threading along with a plurality of threads for engagement with the threaded ring.

In one example, and as shown, the screw gland engages the jack plate, alternatively to engaging the plug/seal retainer, as is found in traditional systems. This teaches away from the traditional art to have a screw gland which doesn't engage the plug/seal retainer. Traditional screw glands are completely designed for the purpose of engaging the plug/seal retainer. The present disclosure of the screw gland improves functionality and design.

### Engagement/Disengagement:

In the arrangement shown, as one example, system 10 is configured to provide ease of assembly or disassembly. The terms engagement and disengagement are utilized herein. Engagement meaning when the jack plate and plurality of jack bolts are engaged with the fluid end of a mud pump. Said another way, the jack plate and jack bolts are engaged when they engage the seal retainer, when the system is activated such that high pressure fluid is then activated in the pump and operation commences.

On the contrary, disengagement occurs when the jack bolt torques are released and/or the jack plate is released. This occurs during maintenance, repairs, or other movement operations where the seal retainer is desired to be removed.

### Secondary Load Holding Capacity:

In the arrangement shown, as one example, the threaded engagement includes a secondary load holding capacity. In this way, any remaining high pressure or unescaped high pressure can escape at a safe rate prior to full disengagement. In this way, a safety feature is hereby contemplated for use.

In one example of a secondary load holding capacity feature, system 10 includes a vent feature created when the load of the jack bolts on the seal are loosened. Said another way, loosening the plurality of jack bolts removes preload on the seal, any residual pressure then vents to the atmosphere when the jack bolts are loosened due to a gap created. Although a gap and/or space is created which provides for venting, the plurality of jack bolts are still secured via a portion of threading.

Furthermore, in the next step, the operator must then unthread the screw gland from the threaded ring. If any residual pressure remains in the fluid end when the jack bolts are loosened, the plug remains safely trapped and/or secured by the screw gland. This safety feature and/or two-step process eliminates any chance that residual pressure remains in the fluid end that could launch the seal retainer out of the pump.

### Axis/Axial Direction:

In the arrangement shown, as one example, the forces of the plurality of jack bolts engaging the jack plate cause an axial force or separation. This axial force is generally seen as a vertical force when applied in a vertical fashion. However, the orientation of the system could be such that the axial force is a horizontal force or a combination thereof. The axial force causes the jack plate to engage the seal retainer. When the axial force is released, via releasing torque on the jack bolts, the jack plate disengages with the seal retainer.

### Cross-Pattern:

In the arrangement shown, as one example, 8 jack bolts are utilized. To ensure even engagement and/or disengagement, a cross pattern of tightening to a predetermined torque and/or release should be best utilized for highest performance and safety precautions. The cross pattern is shown in the figures, but described as tightening a first bolt a predetermined amount, then a second jack bolt, then a third jack bolt nearer the first, then a fourth jack bolt nearer the second, then a fifth jack bolt nearer the third, then a sixth jack bolt nearest the fourth, then a seventh jack bolt nearest the fifth, then an eighth jack bolt nearest the sixth. This sequence can be continued for applications involving additional jack bolts. Furthermore, depending on the tightening, this process may be repeated two times, three times, four times, or more.

In addition to the above identified features, options, controls, and components, system 10 may also include other features and functionalities, among other options, controls, and components.

It will be appreciated by those skilled in the art that other various modifications could be made to the system, process, and method of use without parting from the spirit and scope of this disclosure. All such modifications and changes fall within the scope of the claims and are intended to be covered thereby.

What is claimed:

1. A valve cover assembly, comprising:
a seal;
the seal extending a length from a top to a bottom;
a seal retainer;
the seal retainer having a plurality of threads;
wherein the plurality of threads of the seal retainer are configured to receive the plurality of threads of a stem;
a jack plate;
the jack plate extending a length from a top to a bottom;
the jack plate having a first end and a second end;
the jack plate having a plurality of apertures;
each of the plurality of apertures having a plurality of threads;
the jack plate having a stem aperture;
the stem aperture having a plurality of threads;
a plurality of jack bolts;
each of the plurality of jack bolts extending a length between a top and a bottom;
each of the plurality of jack bolts having threads;
each of the plurality of jack bolts having a cap;
the plurality of jack bolts providing a separation;
wherein the plurality of jack bolts are threaded into the plurality of apertures of the jack plate for engagement;

2. The assembly of claim 1, further comprising:
a stem;
the stem extending a length from a top end to a lower end;
the stem having a plurality of threads;
the stem having a lower shoulder;
wherein the lower end of the stem passes through the jack plate and threads into the seal retainer to maintain concentricity between the seal retainer and the jack plate;
wherein the lower shoulder of the stem serves as a mechanical stop to limit axial movement of the jack plate within the assembly.

3. The assembly of claim 1, further comprising:
wherein the plurality of jack bolts seal the valve cover assembly during engagement; wherein during engagement the plurality of jack bolts are tightened evenly in a cross-pattern to a predefined torque; in this way, the tightening of the plurality of jack bolts creates an axial force which causes the jack plate to separate from the seal retainer, creating a separation; wherein when the separation is created, the seal retainer is in compression, the state of compression creating a seal capable of high pressure operation.

4. The assembly of claim 1, further comprising:
wherein the plurality of jack bolts release the valve cover assembly during disengagement; wherein during disengagement, the plurality of jack bolts are un-torqued evenly in a cross-pattern from a predefined torque; in this way, the releasing of the plurality of jack bolts releases an axial force which causes the jack plate to decrease the separation from the seal retainer; wherein when the separation is released, the compression of the seal retainer is released, the state of compression creating a seal capable of high pressure operation is released.

5. The assembly of claim 1, further comprising:
at least one valve;
the valve having a valve cover;
the valve having a top and a bottom;
a valve guide retainer;
a valve guide;
a lock washer;
a cap screw.

6. The assembly of claim 1, further comprising:
a threaded ring.

7. The assembly of claim 1, further comprising:
a lift eye;
the lift eye extending a length from a top to a bottom;
the lift eye having retaining rings.

8. The assembly of claim 1, further comprising:
a plurality of cap screws.

9. The assembly of claim 1, further comprising:
a screw gland;
the screw gland having a plurality of apertures.

10. A reciprocating pump assembly, comprising:
a reciprocating pump;
the reciprocating pump having a fluid end;
the fluid end having a high pressure fluid;
the fluid end having a high pressure area;
the fluid end having a low pressure area;
the fluid end having a bore;
the reciprocating pump having a power end;
the reciprocating pump having a plurality of blocks;
the reciprocating pump having a plurality of openings;
a plurality of valve covers;
a seal;
the seal extending a length from a top to a bottom;
a seal retainer;
the seal retainer having a plurality of threads;
wherein the plurality of threads of the seal retainer are configured to receive the plurality of threads of a stem;
a jack plate;
the jack plate extending a length from a top to a bottom;
the jack plate having a first end and a second end;
the jack plate having a plurality of apertures;
each of the plurality of apertures having a plurality of threads;
the jack plate having a stem aperture;
the stem aperture having a plurality of threads;
a plurality of jack bolts;
each of the plurality of jack bolts extending a length between a top and a bottom;
each of the plurality of jack bolts having threads;
each of the plurality of jack bolts having a cap;
the plurality of jack bolts providing a separation;
wherein the plurality of jack bolts are threaded into the plurality of apertures of the jack plate for engagement.

11. The assembly of claim 10, further comprising:
a stem;
the stem extending a length from a top end to a lower end;
the stem having a plurality of threads;
the stem having a lower shoulder;
wherein the lower end of the stem passes through the jack plate and threads into the seal retainer to maintain concentricity between the seal retainer and the jack plate;
wherein the lower shoulder of the stem serves as a mechanical stop to limit axial movement of the jack plate within the assembly.

12. The assembly of claim 10, further comprising:
wherein the plurality of jack bolts seal the valve cover assembly during engagement; wherein during engagement the plurality of jack bolts are tightened evenly in a cross-pattern to a predefined torque; in this way, the tightening of the plurality of jack bolts creates an axial force which causes the jack plate to separate from the seal retainer, creating a separation; wherein when the separation is created, the seal retainer is in compression, the state of compression creating a seal capable of high pressure operation.

13. The assembly of claim 10, further comprising:
wherein the plurality of jack bolts release the valve cover assembly during disengagement; wherein during disengagement, the plurality of jack bolts are un-torqued evenly in a cross-pattern from a predefined torque; in this way, the releasing of the plurality of jack bolts releases an axial force which causes the jack plate to decrease the separation from the seal retainer; wherein when the separation is released, the compression of the seal retainer is released, the state of compression creating a seal capable of high pressure operation is released.

14. The assembly of claim 10, further comprising:
at least one valve;
the valve having a valve cover;
the valve having a top and a bottom;
a valve guide retainer;
a valve guide;
a lock washer;
a cap screw.

15. The assembly of claim 10, further comprising:
a threaded ring.

16. The assembly of claim 10, further comprising:
a lift eye;
the lift eye extending a length from a top to a bottom;
the lift eye having retaining rings.

17. The assembly of claim 10, further comprising:
a plurality of cap screws.

18. The assembly of claim 10, further comprising:
a screw gland;
the screw gland having a plurality of apertures.

19. A method of creating a high pressure seal, comprising the steps:
providing a seal retainer having a plurality of threads; wherein the plurality of threads of the seal retainer are configured to receive the plurality of threads of a stem;
providing a jack plate; the jack plate extending a length from a top to a bottom; the jack plate having a first end and a second end; the jack plate having a plurality of apertures;
each of the plurality of apertures having a plurality of threads; the jack plate having a stem aperture; the stem aperture having a plurality of threads;
providing a plurality of jack bolts; each of the plurality of jack bolts extending a length between a top and a bottom; each of the plurality of jack bolts having threads; each of the plurality of jack bolts having a cap;
engaging the plurality of jack bolts are threaded into the plurality of apertures of the jack plate for engagement;
separating the plurality of jack bolts from the seal retainer by applying an axial force by threading the plurality of jack bolts;
creating a high pressure seal by applying the axial force to each of the plurality of jack bolts.

20. The method of claim 19, further comprising the steps:
engaging the plurality of jack bolts evenly; wherein during engagement the plurality of jack bolts are tightened evenly in a cross-pattern to a predefined torque; in this way, the tightening of the plurality of jack bolts creates an axial force which causes the jack plate to separate from the seal retainer, creating a separation; wherein when the separation is created, the seal retainer is in compression, the state of compression creating a seal capable of high pressure operation;
releasing the high pressure seal by disengaging; wherein the plurality of jack bolts release the valve cover assembly during disengagement; wherein during disengagement, the plurality of jack bolts are un-torqued evenly in a cross-pattern from a predefined torque; in this way, the releasing of the plurality of jack bolts releases an axial force which causes the jack plate to decrease the separation from the seal retainer; wherein when the separation is released, the compression of the seal retainer is released, the state of compression creating a seal capable of high pressure operation is released.
